(19) United States
(12) Patent Application Publication
(10) Pub. No.: US 2004/0181128 A1
(43) Pub. Date: Sep. 16, 2004

(54) Determining the geometry and dimensions of a three-dimensional object

(76) Inventor: Martin W. Masters, Hillsborough, NJ (US)

Correspondence Address: Elsa Keller, Siemens Corporation, Intellectual Property Department, 170 Wood Avenue South, Iselin, NJ, 08830, US

(21) Appl. No.: 10/385,587

(22) Filed: Mar. 11, 2003

(51) Int. Cl. A61B 1/267

(52) U.S. Cl. 600/200

(57) ABSTRACT

The output of an distance measuring or imaging assembly positioned in a lumen of fixed, known geometry and size can provide data sufficient to determine the three-dimensional geometry and dimensions of an object such as an ear canal. The lumen's geometry and dimensions may be predetermined or measured by measuring or imaging an object of known geometry and dimensions.

### BACKGROUND AND SUMMARY OF THE INVENTION

A hearing instrument residing partially or wholly in the ear canal of the user requires a shell that can be comfortably inserted and retained in the ear canal. Currently, a variety of labor-intensive techniques such as wax molds are employed to obtain a three-dimensional geometry of the ear canal. Given the increasing use of rapid prototyping and manufacturing technology to fabricate the hearing instrument's shell, an electronic scanning technique that directly yields a digital representation of the ear canal and any desired surrounding structure, or perhaps a representation of the outer ear itself, would be most desirable.

Such a digital representation of the ear canal and the outer ear may be obtained with an apparatus having a flexible distance-measuring or imaging assembly such as an imaging catheter or an endoscopic unit that travels within a lumen of known geometry and size. This apparatus is inserted in the ear canal and then a series of measuring or imaging processes are performed as the assembly is repositioned axially, and perhaps rotationally within the apparatus. The output of the assembly is a set of distances that are correlated with the geometry and dimensions of the lumen to reconstruct the geometry of the scanned area.

### BRIEF DESCRIPTION OF THE DRAWINGS

FIG. 1 is a drawing of an ear and an ear canal with an apparatus for determining the surrounding geometry and dimensions of the inserted therein;

FIG. 2 is a drawing of a section of the ear canal with the apparatus inserted therein;

FIG. 3 is a drawing of an apparatus and a device for determining the three-dimensional geometry and dimensions of the lumen of the apparatus; and

FIG. 4 is a drawing of an apparatus that can produce a three-dimensional representation of an object such as teeth.

### DESCRIPTION OF THE INVENTION

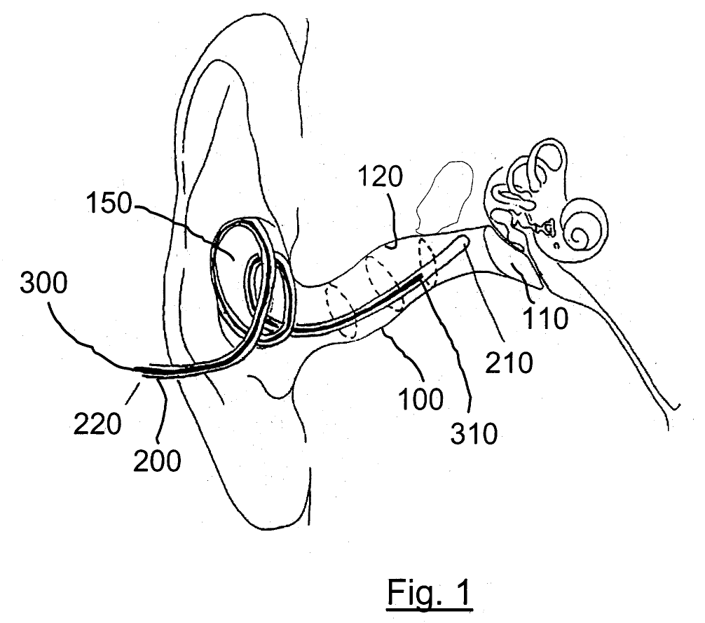

A cross-section of an ear and ear canal 100 is illustrated in FIG. 1. An apparatus 200 having a lumen 220 of known and fixed geometry and dimensions is positioned in the ear canal 100, its tip 210 nearly reaching the tympanic membrane 110. The apparatus 200 may be fabricated from an optically-clear, rigid or semi-rigid, bio-compatible material, such as polycarbonate. A distance-measuring or imaging assembly 300, such as an imaging catheter or an endoscopic unit (hereafter the “imaging assembly 300”), is inserted into the lumen 220, and travels within and through the lumen 220. In addition to axial freedom of motion, the imaging assembly 300 may also rotate within the lumen 220.

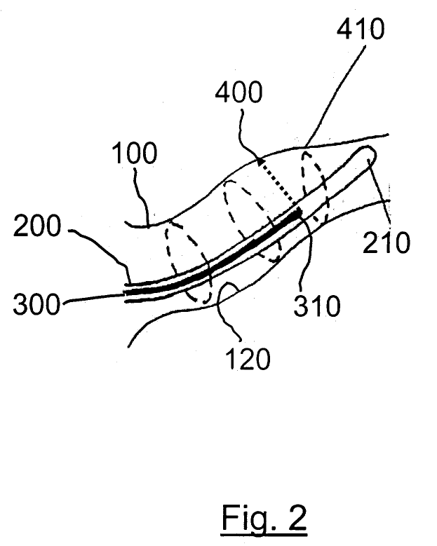

Using a method and apparatus such as that described in U.S. Pat. No. 6,134,003, incorporated herein by reference, the imaging assembly 300 performs a measuring or imaging function, generating an output proportional to the distance from the assembly tip 310 (the point of measurement) to a point 400 on the inner wall 120 of the ear canal 100 (see FIG. 2). Since the geometry and dimensions of the lumen 220 and the position of the assembly tip 310 within the lumen 220 are known, the position of the point 400 relative to the tip 310 can be determined from the output of the assembly 300.

To reconstruct the geometry and dimensions of the ear canal 100, a set of points along the length the inner wall 120 are required. If desired, the process can continue into the concha or bowl 150 of the ear, the portion of the ear outside of the ear canal 100. Referring to FIG. 2, the tip 310 of the assembly 300 is shown at a position a distance away from the apparatus tip 210. Given the particular rotational orientation of the assembly 300 shown in the figure, the imaging assembly 300 measures (or images) the distance to a point 400 on the inner wall 120 of the ear canal 100. If the imaging assembly 300 is rotated, a series of points 410 around the inner wall 120 will be generated. Note that the points 400 comprising the series 410 need not define a closed path but rather, if the imaging assembly 300 is withdrawn simultaneously as it is rotated, the path 410 would be helical or spiral in form.

The imaging assembly 300 may use ultrasound, optical coherence tomography, or any other suitable technology for determining the distance from the assembly 300 to inner wall 120 of the ear canal 100. The catheters described in U.S. Pat. Nos. 6,134,003 and 5,830,145, also incorporated herein by reference, are suitable for use in the imaging assembly 300.

The set of data points for the entire ear canal 100 and optionally at least a portion of the concha 150 is obtained by repositioning the imaging assembly 300 within the lumen 220 after each measuring or imaging process. This repositioning may be achieved by moving the imaging assembly 300 axially after each measurement and perhaps rotating the imaging assembly 300 as well. The assembly 300 could be pulled out of the lumen 220, either step-wise or continuously, resulting in either sliced or helical data. The number of points measured or imaged will determine the resolution achieved and interpolation may be utilized to smooth the data and provide a continuous surface.

The raw data from the imaging assembly 300 is the distance from the tip 310 to the inner wall 120 measured orthogonally at a number of points along the length of the lumen 220. When the rotational orientation of the imaging assembly 300 is coupled with the previously-known three-dimensional geometry and dimensions of the lumen 220 (and therefore the relative location of the assembly tip 310 at any point within the lumen 220), each distance measurement can be converted to a point in space (i.e., in xyz coordinates or any other suitable coordinate system, the origin perhaps being the apparatus tip 210). The conversion can be performed manually or with the aid of a computer program.

The resulting point data in turn may be processed to remove outliers and other unwanted information, such as noise. The remaining data represents a three-dimensional image of the ear canal, concha, and other anatomy, complete or in part as desired. This in turn may be supplied to a rapid prototyping method such as that described in U.S. patent application Ser. No. 09/887,939, filed Jun. 22, 2001, incorporated by reference herein.

The apparatus 200 may have a circular cross-section and may be provided in a variety of sizes and shapes to accommodate different ears. The tip 210 may be closed or open, and the lumen 220 may have straight, bent, spiral, or curved (circular, elliptical, parabolic, or other) sections. Additionally, the apparatus 200 itself (providing a framework for the lumen 220) may have straight, bent, spiral, or curved (circular, elliptical, parabolic, or other) sections, following the shape of the lumen 220.

Instead of using a rigid material for the apparatus 200, the apparatus 200 may be fabricated from a semi-rigid material shaped for the patient's ear. Also, the apparatus 200 could have more than one lumen 220, allowing it to carry more than one imaging assembly 300.

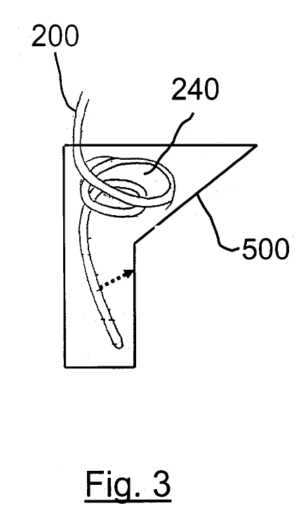

The three-dimensional geometry and dimensions of the lumen 220 may be determined by measuring or imaging an object of known dimensions. For example, the ear canal and the adjacent outer ear might be approximated as a cylinder and a cone (or a truncated cone) attached thereto, respectively. Such an object 500 is shown in FIG. 3. Since the points on the surface of the object 500 may be defined as a set of predetermined xyz coordinates, by working backwards using the distance and the radial orientation of the imaging assembly 300, the geometry and dimensions of the lumen 220 can be determined.

Given the shape of lumen 220, data may be generated for points within the spiral portion 240 of the lumen 220 if the imaging assembly 300 rotates a full 360° and the measuring or imaging continues in that region. In that case, those data points within the spiral portion 240 can be discarded. Alternatively, data collection could cease beyond a predetermined arc of rotation or in the event of a discontinuity in the reflections, which would arise as the reflections sensed by the imaging assembly 300 passes from reflecting off the concha or bowl 150 of the ear (that portion of the ear external to the ear canal 100). As an additional alternative, the assembly 300 could be rotated only a portion of the entire 360°.

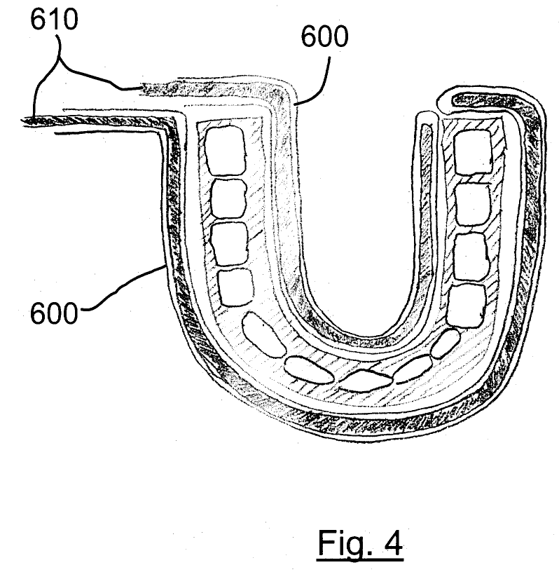

This method of determining geometry and dimensions may be used with any object. Given a set of known points from which measurements are taken, one can determine the three-dimensional geometry and size of an object. In the case of the ear canal discussed, above, the set of known points is obtained with a lumen 220 of known geometry and dimensions. Other objects, such as teeth could also be measured or imaged, given an appropriate device or apparatus containing a channel or lumen for holding and routing the distance-measuring or imaging assemblies. A partial cross-section of such a apparatus 600 is shown in FIG. 4. The apparatus is provided with two assemblies 610 for measuring or imaging the two sides of the semi-circular row of teeth.

What is claimed is:

1. An apparatus, comprising:
at least one lumen of known, fixed geometry and dimensions; and
a distance-measuring or imaging assembly that travels within the lumen.

2. An apparatus as set forth in claim 1, where the assembly has axial and rotational freedom of motion.

3. An apparatus as set forth in claim 1, where the assembly comprises an imaging catheter or an endoscopic unit.

4. An apparatus as set forth in claim 1, where at least a portion of the lumen has a straight section, a bent section, a section having a curvature, or a section spiral in form.

5. An apparatus as set forth in claim 4, where at least a portion of the apparatus has a straight section, a bent section, a section having a curvature, or a section spiral in form.

6. An apparatus as set forth in claim 1, where the apparatus comprises two or more lumens, each lumen comprising a distance-measuring or imaging assembly traveling therein.

7. An apparatus comprising at least one lumen of fixed, known geometry and dimensions within which a distance-measuring or imaging assembly travels.

8. An apparatus, comprising:
at least one lumen of known, fixed geometry and dimensions, where at least a portion of the lumen has a straight section, a bent section, a section having a curvature, or a section spiral in form;
a distance-measuring or imaging assembly traveling within the lumen; and
means for correlating the output of the assembly with the known geometry and dimensions of the lumen.

9. A method for obtaining a three-dimensional digital representation of at least a portion of the ear canal, comprising:
inserting an apparatus, comprising a lumen of known geometry and dimensions, and a distance-measuring or imaging assembly traveling therein, into the area of interest; and
performing two or more measuring or imaging operations.

10. A method as set forth in claim 9, where the step of performing two or more measuring or imaging operations comprises repositioning the assembly within the lumen.

11. A method as set forth in claim 10, where the step of repositioning the assembly within the apparatus comprises
moving the catheter axially within the lumen; and
optionally rotating the catheter within the lumen.

12. A method as set forth in claim 9, further comprising
in response to the step of performing two or more measuring or imaging operations, generating an output; and
correlating the output with the known geometry and dimensions of the lumen.

13. A method for determining the three-dimensional geometry and dimensions of a lumen within an apparatus, comprising:
inserting the apparatus comprising a lumen of unknown geometry and dimensions and a distance-measuring or imaging assembly traveling therein, into an object of known geometry and dimensions;
rotatably withdrawing the assembly from the lumen;
performing measuring or imaging operations as the assembly is withdrawn;
generating an output from the assembly and correlating the output with the known geometry and dimensions of the object; and
calculating the geometry and dimensions of the lumen.

14. A method for obtaining a three-dimensional digital representation of at least a portion of an object, comprising:
positioning an apparatus, comprising a lumen of known geometry and dimensions and a distance-measuring or imaging assembly traveling therein, adjacent to the object; and
performing measuring or imaging operations as the assembly is repositioned within the apparatus.

15. A method for obtaining the relative location of two or more points in space, comprising:
measuring the distance to the points from a distance-measuring or imaging assembly at locations along a fixed-course of known geometry and dimensions.
